SEQUENCE LISTING (3 sequences)

SEQ ID NO: 1 - protein, 494 aa, Homo sapiens
MSTTLLSAFYDVDFLCKTEKSLANLNLNNMLDKKAVGTPVAAAPSSGFAPGFLRRHSASNLHALAHPAPSPGSCSPKFPGAANGSSCGSAAAGGPTSYGTLKEPSGGGGTALLNKENKFRDRSFSENGDRSQHLLHLQQQQKGGGGSQINSTRYKTELCRPFEESGTCKYGEKCQFAHGFHELRSLTRHPKYKTELCRTFHTIGFCPYGPRCHFIHNADERRPAPSGGASGDLRAFGTRDALHLGFPREPRPKLHHSLSFSGFPSGHHQPPGGLESPLLLDSPTSRTPPPPSCSSASSCSSSASSCSSASAASTPSGAPTCCASAAAAAAAALLYGTGGAEDLLAPGAPCAACSSASCANNAFAFGPELSSLITPLAIQTHNFAAVAAAAYYRSQQQQQQQGLAPPAQPPAPPSATLPAGAAAPPSPPFSFQLPRRLSDSPVFDAPPSPPDSLSDRDSYLSGSLSSGSLSGSESPSLDPGRRLPIFSRLSISDD

SEQ ID NO: 2 - DNA, 3702 bp, Homo sapiens
  misc_feature: RefSeq NM_006887 corresponds to GI188536101, which replaced gi 15812177 on May 20, 2008.
gagttccagcagtccgcgagctgccgtcggctccgcggggggggcgggccgggcaccccggggcgcggaggagcgctcctcgcttctctccttcccccctgccgcactccgccggaccctcccgccggcccgcgccgctgcactcgccctctcctctcgccccccggcaaactttcggcccctccccgcccctcgcccgttattcgtcgtggctcaagcccggccacgccgccccaagggctcctcccgacctcccggcctgccgctccggccactgcgggatccagaaacatgtcgaccacacttctgtccgccttctacgatgtcgacttcttgtgcaagacagagaaatccctggccaacctcaacctgaacaacatgctggacaagaaggcggtggggacgcctgtggccgccgcccccagctcgggcttcgcgccgggattcctccgacggcactcggccagcaacctgcatgcactcgcccaccccgcgcccagccccggcagctgctcgcccaagttcccgggcgccgctaacggcagcagctgcggcagcgcggcggccggcggtccgacctcctacggcacccttaaggagccgtcggggggcggcggcacagccctgctcaacaaggagaacaaattccgggaccgctcgtttagcgagaacggcgatcgcagccagcacctcctgcacctgcagcagcagcagaaggggggcggcggctcccagatcaactccacgcgctacaagaccgagctgtgccggcccttcgaggagagcggcacgtgcaagtacggcgaaaagtgccagttcgcgcatggcttccacgagctgcgcagcctgactcgccatccgaagtacaagaccgagctgtgccgcacctttcataccatcggcttctgcccctatgggccgcgctgccacttcatccacaacgcggacgagcggcggcccgcgccgtcggggggcgcctccggggacctgcgtgcctttggcacgcgcgatgcgttgcacctgggcttcccgcgggagccgcggcccaagttgcaccacagcctcagcttctcgggcttcccgtcgggccaccatcagcccccgggcggcctcgagtcgccgctgctgctcgacagccccacgtcgcgcacgccgccgccgccctcctgctcttcggcctcgtcctgctcctcctccgcctcctcctgttcctcggcctccgcggcctccacgccctcgggcgccccgacatgctgcgcctccgcggcggccgcggctgcggccgctctgctgtacggcaccgggggcgccgaggacctgctggcgccgggggccccgtgcgcggcctgctcgtcggcctcgtgcgccaacaacgccttcgccttcggtccggagctcagcagcctcatcacgccgctcgccatccagacccacaactttgccgccgtggccgccgccgcctactaccgcagtcagcagcagcagcagcagcagggcctggcgccccccgcgcagccgccggcgccgcccagcgcgaccctccccgccggggccgccgcacctccctcgccgcccttcagcttccagctgccgcgccgcctgtccgactcgcccgtgttcgacgcgccccccagccccccggactcgctgtcggaccgcgacagctacctaagcggctccctgagctccggcagcctcagcggctctgagtctcccagcctcgaccctggccgccgcctgccaatcttcagccgcctctccatctccgacgactgaggcaagagggcgccagtgaggaggaagggaaggcggttcagagatgttggaggacacccctcgccatctcgcccttgctgggggcacgggagtggggggggtgacatgggccctaggcagactgcaagcccgaccgagcacttggactcgaactctgtgccgggaggggcccccacccctcctttttcggtttcctcttgtctttttttttttatttttattacgaagtttcattctttttgagcaaaaaagtcgaactttttctgttgaacaaaatattcacaacagggcagttgtgatacgaatagaacaaaaaaaaaaaaaaaacacttaaactttgttaggactccgatgagtttgggacttcaggaaaaatcaacccagcaccagcagctaccaaccaccattccatctcttcacttgaacagcattagttaagtccagatgtgggaacccttctcttggaagaagttcctaattgtgtctcagaccggtgtaaacaaaccagccagccgccaccttgctaaacctataagctttttaaaatccaatatattctgccaagaatatgccttgatagttagccctcagcccataggtgttttttgttttttaacagaattatatatgtctgggggtgaaaaaacccttgcattccaaaggtccatactggttacttggtttcattgccaccacttagtggatgttcagtttagaaccattttgtctgctccctctggaagccttgcgcagagcttactttgtaattgttggagaataactgctgaatttttagctgttttgagttgattcgcaccactgcaccacaactcaatatgaaaactatttaacttatttattatcttgtgaaaagtatacaatgaaaattttgttcatactgtatttatcaagtatgatgaaaagcaatagatatatattcttttattatgttaaattatgattgccattattaatcggcaaaatgtggagtgtatgttcttttcacagtaatatatgccttttgtaacttcacttggttattttattgtaaatgagtacaaaattcttaatttaagagattgtatgtaatatttatttcattaatttctttccttgtttacgtaaattttgaaagattgcatgatttcttgacagaaatcgatcttgatgctgtggaagtagtttgaggaacatcctatgagttttcttagaatgtataaaggttgtagcccatccaacttcaaagaaaaaaatgaccacatactttgcaatcaggctgaaatgtggcatgcttttctaattccaactttataaactagcaaaaaagtgtttgcttattccaccagttctactgtgacatactcgagtataaagacatgtagcaataacggggagtggggggggagtctcacagtgcctttggaagggcccgaacttgccttaaatcttcctcaaccaaataagtattttattagtgcttgagagaatctgaatgtaggatgggttcaactgcacaaaaggaaaagatttttaccactttttttatatagatataaagtgaagcaaccgccttagtgctgaaatatgtagtacatgaatatgccttgtttaattacagaaaattccaaaacttgtactatttttttttccatgtagaaaggcaggaatgtctcctaagctttcctggacagcagatgaatgagcggtagctttagtttgtacgtaggtacagttggagcactatatgtactctctggactactttggacagaagtaggtttttgaatgtaacaagataagtcaacttgagttgtaatatattttggggaatcagctcactacaaattgtgactgtaaacattgtactgtaaatgttttgtagttttcccccaataaaatttttgggaaaaaaaggtaaaaaaaaaa

SEQ ID NO: 3 - DNA, 2528 bp, Homo sapiens
  misc_feature: This sequence corresponds to GI15812177, which was replaced by GI188536101 on May 20, 2008.
gagttccagcagtccgcgagctgccgtcggctccgcggggggggcgggccgggcaccccggggcgcggaggagcgctcctcgcttctctccttcccccctgccgcactccgccggaccctcccgccggcccgcgccgctgcactcgccctctcctctcgccccccggcaaactttcggcccctccccgcccctcgcccgttattcgtcgtggctcaagcccggccacgccgccccaagggctcctcccgacctcccggcctgccgctccggccactgcgggatccagaaacatgtcgaccacacttctgtccgccttctacgatgtcgacttcttgtgcaagacagagaaatccctggccaacctcaacctgaacaacatgctggacaagaaggcggtggggacgcctgtggccgccgcccccagctcgggcttcgcgccgggattcctccgacggcactcggccagcaacctgcatgcactcgcccaccccgcgcccagccccggcagctgctcgcccaagttcccgggcgccgctaacggcagcagctgcggcagcgcggcggccggcggtccgacctcctacggcacccttaaggagccgtcggggggcggcggcacagccctgctcaacaaggagaacaaattccgggaccgctcgtttagcgagaacggcgatcgcagccagcacctcctgcacctgcagcagcagcagaaggggggcggcggctcccagatcaactccacgcgctacaagaccgagctgtgccggcccttcgaggagagcggcacgtgcaagtacggcgaaaagtgccagttcgcgcatggcttccacgagctgcgcagcctgactcgccatccgaagtacaagaccgagctgtgccgcacctttcataccatcggcttctgcccctatgggccgcgctgccacttcatccacaacgcggacgagcggcggcccgcgccgtcggggggcgcctccggggacctgcgtgcctttggcacgcgcgatgcgttgcacctgggcttcccgcgggagccgcggcccaagttgcaccacagcctcagcttctcgggcttcccgtcgggccaccatcagcccccgggcggcctcgagtcgccgctgctgctcgacagccccacgtcgcgcacgccgccgccgccctcctgctcttcggcctcgtcctgctcctcctccgcctcctcctgttcctcggcctccgcggcctccacgccctcgggcgccccgacatgctgcgcctccgcggcggccgcggctgcggccgctctgctgtacggcaccgggggcgccgaggacctgctggcgccgggggccccgtgcgcggcctgctcgtcggcctcgtgcgccaacaacgccttcgccttcggtccggagctcagcagcctcatcacgccgctcgccatccagacccacaactttgccgccgtggccgccgccgcctactaccgcagtcagcagcagcagcagcagcagggcctggcgccccccgcgcagccgccggcgccgcccagcgcgaccctccccgccggggccgccgcacctccctcgccgcccttcagcttccagctgccgcgccgcctgtccgactcgcccgtgttcgacgcgccccccagccccccggactcgctgtcggaccgcgacagctacctaagcggctccctgagctccggcagcctcagcggctctgagtctcccagcctcgaccctggccgccgcctgccaatcttcagccgcctctccatctccgacgactgaggcaagagggcgccagtgaggaggaagggaaggcggttcagagatgttggaggacacccctcgccatctcgcccttgctgggggcacgggagtggggggggtgacatgggccctaggcagactgcaagcccgaccgagcacttggactcgaactctgtgccgggaggggcccccacccctcctttttcggtttcctcttgtctttttttttttatttttattacgaagtttcattctttttgagcaaaaaagtcgaactttttctgttgaacaaaatattcacaacagggcagttgtgatacgaatagaacaaaaaaaaaaaaaaaacacttaaactttgttaggactccgatgagtttgggacttcaggaaaaatcaacccagcaccagcagctaccaaccaccattccatctcttcacttgaacagcattagttaagtccagatgtgggaacccttctcttggaagaagttcctaattgtgtctcagaccggtgtaaacaaaccagccagccgccaccttgctaaacctataagctttttaaaatccaatatattctgccaagaatatgccttgatagttagccctcagcccataggtgttttttgttttttaacagaattatatatgtctgggggtgaaaaaacccttgcattccaaaggtccatactggttacttggtttcattgccaccacttagtggatgttcagtttagaaccattttgtctgctccctctggaagccttgcgca

(19) United States
(12) Patent Application Publication
(10) Pub. No.: US 2011/0092428 A1
(43) Pub. Date: Apr. 21, 2011

(54) DETECTING AND CONTROLLING ABNORMAL HEMATOPOIESIS

(75) Inventors: Deborah J. Stumpo, Durham, NC (US); Perry J. Blackshear, Chapel Hill, NC (US)

(73) Assignee: The United States of America,as represented by the Secretary ,Department of Healthyand Human Service, Beathesda, MD (US)

(21) Appl. No.: 12/667,287

(22) PCT Filed: Jul. 1, 2008
(86) PCT No.: PCT/US2008/068900

§ 371 Date: Jan. 21, 2010

Related U.S. Application Data
(60) Provisional application No. 60/947,593, filed on Jul. 2, 2007.

(51) Int. Cl. A61K 38/16; C12Q 1/68; A61P 35/00; A61P 35/02; A61P 7/00

(52) U.S. Cl. 514/13.5; 435/6; 514/19.3; 514/19.6

(57) ABSTRACT

A method of detecting abnormal hematopoiesis in a subject based on abnormal expression of ZFP36L2, a method of controlling hematopoiesis in a subject altering the level or activity of ZFP36L2 protein in the subject, a method of screening for compounds that modulate hematopoiesis based on changes to ZFP36L2 expression, and compounds identified thereby.

### BACKGROUND OF THE INVENTION

Hematopoietic disorders include conditions in which there is insufficient or excessive blood cell production. Such disorders can have many causes, and are frequently secondary conditions to other primary disorders. Although methods of treating hematopoietic disorders and related conditions exist, some such methods have been associated with adverse side effects and health risks. Thus, there remains a desire for additional methods and compounds useful for treating, detecting, monitoring, or controlling hematopoietic disorders and conditions associated therewith.

### BRIEF SUMMARY OF THE INVENTION

The invention provides a method of detecting abnormal hematopoiesis, or a condition associated therewith, in a subject. According to one aspect of the invention, the method comprises detecting abnormal expression of Zinc Finger Protein 36 C3H type-like 2 (ZFP36L2) in the subject, wherein abnormal ZFP36L2 expression in the subject indicates abnormal hematopoiesis or a condition associated therewith. According to another aspect of the invention, the method comprises detecting a mutation in a ZFP36L2 gene in the subject, wherein a mutation in the ZFP36L2 gene in the subject indicates abnormal hematopoiesis or a condition associated therewith.

The invention also provides a method of screening for a compound that modulates hematopoiesis comprising (a) administering a test compound to a cell that expresses ZFP36L2, and (b) detecting a change in ZFP36L2 expression in the cell in the presence of the test compound as compared to a control, wherein a change in ZFP36L2 expression in the cell indicates that the test compound is likely to modulate hematopoiesis.

The invention further provides a method of controlling hematopoiesis in a subject comprising adjusting the level or activity of ZFP36L2 protein in the subject.

### BRIEF DESCRIPTION OF THE DRAWINGS

FIG. 1 is a graph of the number of white blood cells (WBC), red blood cells (RBC), spun hematocrit (Spun HCT) (expressed as a percentage), and platelets in control (+/+), partial ZFP36L2 knock-out (+/−), and complete ZFP36L2 knock-out (−/−) mice.

FIG. 2 is a graph of the number of white blood cells (WBC), neutrophils, lymphocytes, monocytes, and eosinophils in control (+/+), partial ZFP36L2 knock-out (+/−), and complete ZFP36L2 knock-out (−/−) mice.

FIG. 3 is a graph of the number of hematopoietic progenitor cells in fetal liver at embryonic day 14.5 in control (+/+), partial ZFP36L2 knock-out (+/−), and complete ZFP36L2 knock-out (−/−) mice.

FIG. 4 is a graph of the number of hematopoietic progenitor cells in the yolk sac at embryonic day 11.5 in control (+/+), partial ZFP36L2 knock-out (+/−), and complete ZFP36L2 knock-out (−/−) mice.

FIG. 5 is a graph of the number of hematopoietic progenitor cells in the aorta-gonad-mesonephros (AGM) region at embryonic day 11.5 in control (+/+), partial ZFP36L2 knock-out (+/−), and complete ZFP36L2 knock-out (−/−) mice.

FIG. 6 is a graph of the percent engraftment of repopulated fetal liver cells at one and two months post-engraftment in normal mice (+/+) and complete ZFP36L2 knock-out mice (−/−).

### DETAILED DESCRIPTION OF THE INVENTION

Zinc finger protein 36 like type-2 (ZFP36L2, also known as BRF2, ERF2, ERF-2, TIS11D, and RNF162C) belongs to a family of zinc finger proteins containing tandem zinc-binding motifs characterized by three cysteines followed by one histidine (CCCH). Through the zinc fingers, these proteins can bind to mRNAs containing class II AU-rich elements, generally in their 3′-untranslated regions, followed by degradation of the target mRNA. Without wishing to be bound by any particular theory, it is believed that ZFP36L2 interacts with mRNA species encoding one or more proteins involved in hematopoiesis, and that increased or decreased expression of ZFP36L2 thereby modulates hematopoiesis. The human genomic sequence encoding the ZFP36L2 protein is located approximately at positions 22271678-22264639 of chromosome 2, locus NT_{—}22184.14. The ZFP36L2 protein and mRNA sequences are associated with RefSeq accession numbers NP_{—}008818 (protein) and NM_{—}006887 (mRNA), respectively. The corresponding sequences of other species are known in the art.

In one embodiment, the invention provides a method of detecting abnormal hematopoiesis, or a condition associated therewith, in a subject by detecting abnormal ZFP36L2 expression in the subject, wherein abnormal ZFP36L2 expression in the subject indicates abnormal hematopoiesis or a condition associated therewith. More specifically, abnormally high ZFP36L2 expression indicates abnormally high hematopoiesis or a condition associated therewith, and abnormally low ZFP36L2 indicates abnormally low hematopoiesis or a condition associated therewith.

The subject typically will be an animal, such as a mammal, preferably a human, in which case abnormal ZFP36L2 expression can be detected in a suitable sample from the subject, such as a body fluid (e.g., blood) or tissue sample. However, the subject also can be an isolated cell (e.g., cell culture) as might be useful in the context of research. For instance, the cell can be a hematopoietic or stromal cell, or a different cell of a type that normally expresses ZFP36L2. Suitable cells also include those that have been engineered to express ZFP36L2 or contain a mutated ZFP36L2.

Abnormal hematopoiesis in a given subject encompasses levels of hematopoiesis that are higher or lower, generally to a clinically significant degree, than the levels of hematopoiesis considered “normal” in a given type of subject. Guidance as to normal and abnormal levels of hematopoiesis in a subject can be ascertained by one of ordinary skill in the art. With respect to human subjects, such information is available, for example, by consulting the Physicians' Desk Reference, 61^{st} ed. Montvale, N.J. (2007).

Conditions associated with abnormally high hematopoiesis include, without limitation, polycythemia, cancers or tumors including leukemia, or a combination thereof. Conditions associated with abnormally low hematopoiesis include, without limitation, anemia, thrombocytopenia, myelodysplastic syndrome, or a combination thereof. Furthermore, any of the foregoing conditions can be primary conditions, or can be secondary to other conditions, such as cancer, cancer chemotherapy, infections, dialysis, etc. Conditions associated with abnormal hematopoiesis also include, for the purposes of the invention, a predisposition to developing abnormal hematopoiesis or any condition related thereto. The hematopoiesis is typically of the myeloid lineage.

Abnormal expression of ZFP36L2 in a subject means expression of ZFP36L2 that is higher or lower than the expression of ZFP36L2 in a normal, non-diseased subject of the same type. Generally, abnormal expression of ZFP36L2 in a given subject will be significantly higher or lower than that of a normal non-diseased subject, such that it causes a physiological or phenotypic effect.

Abnormal ZFP36L2 expression can be detected by any method. Generally, abnormal ZFP36L2 expression is detected by comparing the level of ZFP36L2 expression in the subject to a control. The control can be, for example, the level of ZFP36L2 expression in a normal, non-diseased subject of the same type, or a pre-established standard that represents the normal expression level of ZFP36L2 in such a subject.

ZFP36L2 expression can be detected and compared on any basis. For example, abnormal ZFP36L2 expression can be detected on the basis of the level or activity of ZFP36L2 protein. Any technique for detecting, quantifying, and/or comparing protein levels or activities can be used including, without limitation, protein immunostaining, immunoprecipitation, western blot, spectroscopy, enzyme assay, chromatography, Bradford protein assay, and gel electrophoresis techniques.

Immunostaining is a general term in biochemistry that applies to any use of an antibody-based method to detect a specific protein in a sample. Immunohistochemistry or IHC staining of cells or tissue sections is perhaps the most commonly applied immunostaining technique. While the first cases of IHC staining used fluorescent dyes (see immunofluorescence), other non-fluorescent methods using enzymes such as peroxidase (see immunoperoxidase staining) and alkaline phosphatase are now used. These enzymes are capable of catalyzing reactions that give a colored product that is easily detectable by light microscopy. Alternatively, radioactive elements can be used as labels, and the immunoreaction can be visualized by autoradiography.

Immunoprecipitation (IP) is the technique of precipitating an antigen out of solution using an antibody specific to that antigen. This process can be used to enrich a given protein to some degree of purity. Co-immunoprecipitation (also known as a ‘pull-down’) can identify interacting proteins or protein complexes present in cell extracts: by precipitating one protein believed to be in a complex, additional members of the complex are captured as well and can be identified. The protein complexes, once bound to the specific antibody, are removed from the bulk solution by capture with an antibody-binding protein attached to a solid support such as an agarose bead. After washing, the precipitated proteins are eluted and analyzed using gel electrophoresis, mass spectrometry, western blotting, or any number of other methods for identifying constituents in the complex.

The Bradford assay, a colorimetric protein assay, is based on an absorbance shift in the dye Coomassie when bound to arginine and hydrophobic amino acid residues present in protein. The (bound) form of the dye is blue and has an absorption spectrum maximum historically held to be at 595 nm. The anionic (unbound) forms are green and red. The increase of absorbance at 595 nm is proportional to the amount of bound dye, and thus to the amount (concentration) of protein present in the sample. Unlike other protein assays, the Bradford protein assay is less susceptible to interference by various chemicals that may be present in protein samples.

Gel electrophoresis is also useful for the detection of protein in a sample. Proteins can have different charges and complex shapes, and therefore they may not migrate into the gel at similar rates, or at all, when placing a negative to positive EMF on the sample. Proteins therefore, are usually denatured in the presence of a detergent such as sodium dodecyl sulfate/sodium dodecyl phosphate (SDS/SDP) that coats the proteins with a negative charge. Generally, the amount of SDS bound is relative to the size of the protein (usually 1.4 g SDS per gram of protein), so that the resulting denatured proteins have an overall negative charge, and all the proteins have a similar charge to mass ratio. Since denatured proteins act like long rods instead of having a complex tertiary shape, the rate at which the resulting SDS coated proteins migrate in the gel is relative only to its size and not its charge or shape. Proteins are usually analyzed by sodium dodecyl sulfate polyacrylamide gel electrophoresis (SDS-PAGE), by native gel electrophoresis, by quantitative preparative native continuous polyacrylamide gel electrophoresis (QPNC-PAGE), or by 2-D electrophoresis.

A western blot (also called an immunoblot) is a method to detect a specific protein in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Abnormal ZFP36L2 expression also can be detected on the basis of mRNA levels. Suitable techniques for determining the presence and level of expression of ZFP36L2 mRNA in cells are within the skill in the art. For example, total cellular RNA can be purified from a sample by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters by, e.g., the so-called “Northern” blotting technique. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference. For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

Probes can be labeled to high specific activity by either the nick translation method of Rigby et al, J. Mol. Biol., 113:237-251 (1977) or by the random priming method of Fienberg, Anal. Biochem., 132:6-13 (1983), the entire disclosures of which are herein incorporated by reference. The latter can be a method for synthesizing ³²P-labeled probes of high specific activity from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of RNA levels. Using another approach, RNA levels can be quantified by computerized imaging systems, such the Molecular Dynamics 400-B 2D Phosphorimager (Amersham Biosciences, Piscataway, N.J.).

Where radionuclide labeling of DNA or RNA probes is not practical, the random-primer method can be used to incorporate an analogue, for example, the dTTP analogue 5-(N-(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA blotting hybridization techniques, determining the levels of RNA transcript can be accomplished using in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference.

RNA transcript levels can be determined using arrays, e.g., microarrays or gene chips, which include a plurality of nucleic acid probes coupled to the surface of a substrate in different known locations. The arrays include one or more substrate-coupled probes capable of binding to and quantifying ZFP36L2 mRNA transcripts. Microarrays have been generally described in the art in, for example, U.S. Pat. Nos. 5,143,854; 5,242,974; 5,252,743; 5,324,633; 5,384,261; 5,424,186; 5,445,934; 5,451,683; 5,482,867; 5,491,074; 5,527,681; 5,550,215; 5,571,639; 5,578,832; 5,593,839; 5,599,695; 5,624,711; 5,631,734; 5,677,195; 5,744,305; 5,795,716; 5,800,992; 5,831,070; 5,837,832; 5,856,101; 5,858,659; 5,936,324; 5,968,740; 5,974,164; 5,981,185; 5,981,956; 6,025,601; 6,033,860; 6,040,193; 6,090,555; and 6,410,229, U.S. Patent Application Publication No. 20030104411, and Fodor et al., Science, 251: 767-777 (1991). Each of these references is incorporated by reference herein in their entirety.

The relative number of mRNA transcripts in cells also can be determined by reverse transcription of the mRNA transcripts, followed by amplification of the reverse-transcribed transcripts by polymerase chain reaction (RT-PCR). The levels of mRNA transcripts can be quantified in comparison with an internal standard, for example, the level of mRNA from a standard gene present in the same sample. A suitable gene for use as an internal standard includes, e.g., myosin or glyceraldehyde-3-phosphate dehydrogenase (G3PDH). The methods for quantitative RT-PCR and variations thereof are within the skill in the art.

In another aspect, the method of detecting abnormal hematopoiesis comprises detecting a mutation in a ZFP36L2 gene in a subject, wherein a mutation in the ZFP36L2 gene indicates abnormal hematopoiesis or a condition associated therewith. The mutation can be any mutation that interferes with the proper function of ZFP36L2 mRNA or its protein product, for instance, mutations that result in decreased transcription of the ZFP36L2 gene, reduced stability of ZFP36L2 mRNA, or a mutant ZFP36L2 protein with reduced activity as compared to non-mutant ZFP36L2 protein. Such mutations include, for purposes of illustration, those which introduce a premature stop codon (PTC) into the ZFP36L2 mRNA. Mutations in the ZFP36L2 gene can be detected by any technique, such as by detecting differences between the ZFP36L2 gene of a subject and that of a known “normal” ZPF36L2 gene.

The method of detecting hematopoiesis in a subject can be used for any purpose. Non-limiting examples of such uses include the screening or diagnosis of hematopoiesis or conditions associated therewith, the evaluation and development of treatments for such disorders and conditions, and research related to the mechanisms of such disorders and conditions and the discovery of new treatments for such disorders and conditions. The method also can be used in conjunction with a method of controlling hormonal hematopoiesis or treating a condition associated therewith. Such methods include those known in the art and methods described herein.

The invention also provides a method of screening for a compound that modulates hematopoiesis. The method comprises (a) administering a test compound to a cell that expresses ZFP36L2, and (b) detecting a change in ZFP36L2 expression in the cell that expresses ZFP36L2 as compared to a control. A change in ZFP36L2 expression in the cell that expresses ZFP36L2 as compared to the control indicates that the test compound is likely to modulate hematopoiesis. The method also can be used in conjunction with a method of controlling abnormal hematopoiesis or treating a condition associated with abnormal hematopoiesis. Such methods include those known in the art and methods described herein.

Any cell that expresses ZFP36L2 can be used, including cells that endogenously express ZFP36L2 and cells that have been engineered to express ZFP36L2. Suitable cell types include, for example, hematopoietic cells and stromal cells, or any type of cell that can be stably transfected to express ZFP36L2.

A change in expression of ZFP36L2 can be detected by any suitable method, for example, by detecting a change in the level of ZFP36L2 transcription, the level of ZFP36L2 mRNA, or the level or activity of ZFP36L2 protein in the cell. Furthermore, such levels can be directly detected, or indirectly detected using various markers or tags. By way of illustration, the cell can comprise a nucleic acid construct comprising a nucleic acid encoding ZFP36L2 fused to a nucleic acid encoding a marker protein, wherein a change in the expression of ZFP36L2 is detected by detecting a change in the expression of the marker protein. Such a nucleic acid encoding a marker protein can be fused, for instance, to the 3′ end of a ZFP36L2 mRNA, whereby changes to ZFP36L2 mRNA levels can be detected on the basis of the marker protein. Alternatively, the nucleic acid encoding the marker protein can be fused to the ZFP36L2 gene promoter, such that expression of the marker protein is driven by ZFP36L2 transcription, and changes to ZFP36L2 transcription levels can be detected on the basis of the marker protein. Suitable marker proteins include, without limitation, green fluorescence protein (GFP), luciferase, beta-galactosidase, and others known in the art. Specific protocols for using such marker proteins are known in the art, some of which are discussed herein with respect to other aspects of the invention.

The control can be any control that provides an acceptable baseline by which to compare ZFP36L2 expression and detect a change in such expression. For example, the control can be ZFP36L2 expression in the cell, or a cell of the same type, in the absence of the test compound. Alternatively, the control can be a pre-established baseline ZFP36L2 expression level (e.g., pertaining to a given cell type).

The invention is not limited with respect to any particular class or type of test compound. Rather, any test compound can be used, including, without limitation, RNA, DNA, peptides, peptidomimetics, antibodies and fragments thereof, and organic small molecules.

Without wishing to be bound by any particular theory, it is believed that compounds likely to modulate hematopoiesis are those which bind to a ZFP36L2 DNA (e.g., the ZFP36L2 promoter), ZFP36L2 mRNA, or ZFP36L2 protein. Thus, the method of screening for a compound that modulates hematopoiesis can further comprise, prior to administering a test compound to a cell that expresses ZFP36L2, selecting a test compound that binds to (a) ZFP36L2 mRNA, (b) ZFP36L2 protein, or (c) ZFP36L2 promoter. Such selection can be performed by any suitable technique, such as by immobilizing one or more of (a)-(c) on a substrate, contacting the substrate with a test compound or library of test compounds, and detecting binding between a test compound and the immobilized DNA, mRNA, or protein. Alternatively, any one or more of (a)-(c) can be used to pan or scan a library of test compounds (e.g., immobilized test compounds), and compounds that bind (a)-(c) can be selected. Specific protocols for such binding assays are known in the art.

The invention further provides a compound identified by the method of screening described herein, as well as compositions comprising such compounds, which are useful for modulating ZFP36L2 expression and hematopoiesis in a subject.

In another embodiment, the invention provides a method of controlling hematopoiesis in a subject by adjusting the level or activity of ZFP36L2 protein in the subject. The level or activity of ZFP36L2 can be increased or decreased as desired based on the hematopoietic condition of the subject and result sought. For instance, in a subject with a disease or condition associated with abnormally low hematopoiesis, it generally is desirable to increase the level or activity of ZFP36L2 protein, thereby increasing hematopoiesis. In contrast, in subject with a disease or condition associated with abnormally high hematopoiesis, it generally is desirable to reduce the level or activity of ZFP36L2 protein, thereby reducing hematopoiesis. By controlling hematopoiesis in a subject with abnormally high or low hematopoiesis, the abnormal hematopoietic condition and other conditions associated therewith can be treated or the symptoms of such conditions relieved in whole or in part.

The level or activity ZFP36L2 protein can be adjusted by any method. For example, the level or activity of ZFP36L2 protein can be increased or decreased by (a) increasing or decreasing transcription of a nucleic acid encoding ZFP36L2, (b) increasing or decreasing the stability of ZFP36L2 mRNA, (c) increasing or decreasing cellular synthesis of ZFP36L2, (d) increasing or decreasing cellular degradation of ZFP36L2 protein or mRNA, and (e) combinations thereof. Alternately, or in combination, exogenous ZFP36L2 protein can be administered to the subject to increase ZFP36L2 protein levels, for example, by introducing an exogenous nucleic acid into the subject, or cells of the subject, which encodes ZFP36L2. Finally, other compounds that modulate ZFP36L2 or hematopoiesis, such as compounds identified by the methods of screening for such compounds described herein, can be administered to the subject to control hematopoiesis.

Methods of altering gene expression and mRNA stability and translation are well known to those of ordinary skill in the art and include the use of antisense, microRNA, siRNA, naked nucleic acids, and expression systems.

Any stage of gene expression may be modulated, from transcription to post-translational modification. For example, expression of a given gene can be inhibited by inducing RNA interference of the gene with an isolated double-stranded RNA (“dsRNA”) molecule which has at least 90%, for example 95%, 98%, 99% or 100%, sequence homology with at least a portion of the gene product. In a preferred embodiment, the dsRNA molecule is a “short or small interfering RNA” or “siRNA.”

siRNA useful in the present methods comprise short double-stranded RNA from about 17 nucleotides to about 29 nucleotides in length, preferably from about 19 to about 25 nucleotides in length. The siRNA comprise a sense RNA strand and a complementary antisense RNA strand annealed together by standard Watson-Crick base-pairing interactions (hereinafter “base-paired”). The sense strand comprises a nucleic acid sequence which is substantially identical to a nucleic acid sequence contained within the target gene product.

As used herein, the siRNA is “substantially identical” to a target sequence contained within the target nucleic sequence, is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA can also be altered RNA that differs from naturally-occurring RNA by the addition, deletion, substitution and/or alteration of one or more nucleotides. Such alterations can include addition of non-nucleotide material, such as to the end(s) of the siRNA or to one or more internal nucleotides of the siRNA, or modifications that make the siRNA resistant to nuclease digestion, or the substitution of one or more nucleotides in the siRNA with deoxyribonucleotides. Chemically modified siRNAs directed to ZFP36L2 are commercially available, e.g., as SILENCER siRNA from Applied Biosystems (Foster City, Calif.) and STEALTH siRNA from Invitrogen (Carlsbad, Calif.).

One or both strands of the siRNA can also comprise a 3′ overhang. As used herein, a “3′ overhang” refers to at least one unpaired nucleotide extending from the 3′-end of a duplexed RNA strand. Thus, in one embodiment, the siRNA comprises at least one 3′ overhang of from 1 to about 6 nucleotides (which includes ribonucleotides or deoxyribonucleotides) in length, preferably from 1 to about 5 nucleotides in length, more preferably from 1 to about 4 nucleotides in length, and particularly preferably from about 2 to about 4 nucleotides in length. In a preferred embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated gene products. Exemplary methods for producing and testing dsRNA or siRNA molecules are described in U.S. Published Patent Application No. 2002/0173478 and U.S. Pat. No. 7,148,342, the entire disclosures of which are herein incorporated by reference.

Expression of a given gene can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a gene product. Preferably, the antisense nucleic acid comprises a nucleic acid sequence that is 50-100% complementary, more preferably 75-100% complementary, and most preferably 95-100% complementary to a contiguous nucleic acid sequence in an gene product.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators such as acridine or the inclusion of one or more nuclease-resistant groups.

Antisense nucleic acids can be produced chemically or biologically, or can be expressed from a recombinant plasmid or viral vector, as described above for the isolated gene products. Exemplary methods for producing and testing are within the skill in the art; see, e.g., Stein, Science, 261:1004 (1993) and U.S. Pat. No. 5,849,902 to Woolf et al., the entire disclosures of which are herein incorporated by reference.

Expression of a given gene also can be inhibited by an enzymatic nucleic acid. As used herein, an “enzymatic nucleic acid” refers to a nucleic acid comprising a substrate binding region that has complementarity to a contiguous nucleic acid sequence of a gene product, and which is able to specifically cleave the gene product. Preferably, the enzymatic nucleic acid substrate binding region is 50-100% complementary, more preferably 75-100% complementary, and most preferably 95-100% complementary to a contiguous nucleic acid sequence in a gene product. The enzymatic nucleic acids can also comprise modifications at the base, sugar, and/or phosphate groups. An exemplary enzymatic nucleic acid for use in the present methods is a ribozyme.

The enzymatic nucleic acids can be produced chemically or biologically, or can be expressed from a recombinant plasmid or viral vector, as described above for the isolated gene products. Exemplary methods for producing and testing dsRNA or siRNA molecules are described in Werner, Nucl. Acids Res., 23:2092-96 (1995); Hammann, Antisense and Nucleic Acid Drug Dev., 9:25-31 (1999); and U.S. Pat. No. 4,987,071, the entire disclosures of which are herein incorporated by reference.

Gene expression can also be affected by administering expression systems to the subject that enhance or repress expression of the gene product. The expression systems can include genes, promoters, enhancers, repressors, etc., and such techniques are well known within the art. Preferably, the cells of the subject are transfected with a plasmid or viral vector comprising sequences encoding at least one gene product (e.g., ZPF36L2 protein) or gene expression inhibiting composition.

Transfection methods for eukaryotic cells are well known in the art, and include, e.g., direct injection of the nucleic acid into the nucleus or pronucleus of a cell; electroporation; liposome transfer or transfer mediated by lipophilic materials; receptor mediated nucleic acid delivery, bioballistic or particle acceleration; calcium phosphate precipitation, and transfection mediated by viral vectors.

For example, cells can be transfected with a liposomal transfer composition, e.g., DOTAP (N-[1-(2,3-dioleoyloxy)propyl]-N,N,N-trimethyl-ammonium methylsulfate, Boehringer-Mannheim) or an equivalent, such as LIPOFECTIN. The amount of nucleic acid used is not critical to the practice of the invention; acceptable results may be achieved with 0.1-100 micrograms of nucleic acid/10⁵ cells. For example, a ratio of about 0.5 micrograms of plasmid vector in 3 micrograms of DOTAP per 10⁵ cells can be used.

Also, compounds identified by the method of screening of the invention can be administered to a subject in an amount effective to modulate ZFP36L2 expression or hematopoiesis. One skilled in the art can readily determine an effective amount of a stimulating or inhibiting composition to be administered to a given subject, by taking into account factors such as the size and weight of the subject; the extent of disease penetration; the age, health and sex of the subject; the route of administration; and whether the administration is regional or systemic.

One skilled in the art can also readily determine an appropriate dosage regimen for administering a composition. For example, the composition can be administered to the subject once (e.g. as a single injection or deposition). Alternatively, the composition can be administered once or twice daily to a subject for a period of from about three to about one month. In certain instances, the subject may be required to take composition on a long term basis, that is, for weeks, months, years, or indefinitely. Where a dosage regimen comprises multiple administrations, it is understood that the effective amount of the composition administered to the subject can comprise the total amount of composition administered over the entire dosage regimen.

The composition can also be administered to a subject by any suitable enteral or parenteral administration route. Suitable enteral administration routes for the present methods include, e.g., oral, rectal, or intranasal delivery. Suitable parenteral administration routes include, e.g., intravascular administration (e.g., intravenous bolus injection, intravenous infusion, intra-arterial bolus injection, intra-arterial infusion and catheter instillation into the vasculature); peri- and intra-tissue injection; subcutaneous injection or deposition, including subcutaneous infusion (such as by osmotic pumps); direct application to the tissue of interest, for example by a catheter or other placement device (e.g., a retinal pellet or a suppository or an implant comprising a porous, non-porous, or gelatinous material); and inhalation.

In the present methods, the composition can be administered to the subject either as naked RNA, in combination with a delivery reagent, or as a nucleic acid (e.g., a recombinant plasmid or viral vector) comprising sequences that express the gene product or expression inhibiting composition. Suitable delivery reagents include, e.g., the Mirus Transit TKO lipophilic reagent; lipofectin; lipofectamine; cellfectin; polycations (e.g., polylysine), and liposomes.

Recombinant plasmids and viral vectors comprising sequences that express the gene expression inhibiting compositions, and techniques for delivering such plasmids and vectors to cancer cells, are discussed above.

In a preferred embodiment, liposomes are used to deliver a gene expression-inhibiting composition (or nucleic acids comprising sequences encoding them) to a subject. Liposomes can also increase the blood half-life of the gene products or nucleic acids.

Liposomes suitable for use in the invention can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka, Ann. Rev. Biophys. Bioeng., 9:467 (1980); and U.S. Pat. Nos. 4,235,871, 4,501,728, 4,837,028, and 5,019,369, the entire disclosures of which are herein incorporated by reference.

The liposomes for use in the present methods can comprise a ligand molecule that targets the liposome to cancer cells. Ligands which bind to receptors prevalent in cancer cells, such as monoclonal antibodies that bind to tumor cell antigens, are preferred.

The compositions of the present invention may include a pharmaceutically acceptable carrier. The term “pharmaceutically-acceptable carrier” as used herein means one or more compatible solid or liquid fillers, diluents, other excipients, or encapsulating substances which are suitable for administration into a human or veterinary patient. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner so as not to substantially impair the desired pharmaceutical efficacy. “Pharmaceutically acceptable” materials are capable of administration to a patient without the production of undesirable physiological effects such as nausea, dizziness, rash, or gastric upset. It is, for example, desirable for a therapeutic composition comprising pharmaceutically acceptable excipients not to be immunogenic when administered to a human patient for therapeutic purposes.

The pharmaceutical compositions may contain suitable buffering agents, including: acetic acid in a salt; citric acid in a salt; boric acid in a salt; and phosphoric acid in a salt. The pharmaceutical compositions also may contain, optionally, suitable preservatives, such as: benzalkonium chloride, chlorobutanol, parabens and thimerosal.

The pharmaceutical compositions may conveniently be presented in unit dosage form and may be prepared by any of the methods well known in the art of pharmacy. All methods include the step of bringing the active agent into association with a carrier that constitutes one or more accessory ingredients. In general, the compositions are prepared by uniformly and intimately bringing the active composition into association with a liquid carrier, a finely divided solid carrier, or both, and then, if necessary, shaping the product.

Compositions suitable for parenteral administration conveniently comprise a sterile aqueous preparation of the inventive composition, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. which is incorporated herein in its entirety by reference thereto.

The delivery systems of the invention are designed to include time-released, delayed release or sustained release delivery systems such that the delivering of the inventive composition occurs prior to, and with sufficient time, to cause sensitization of the site to be treated. The inventive composition may be used in conjunction with other therapeutic agents or therapies. Such systems can avoid repeated administrations of the inventive composition, increasing convenience to the subject and the physician, and may be particularly suitable for certain compositions of the present invention.

Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drags are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. In addition, pump-based hardware delivery systems can be used, some of which are adapted for implantation.

The following example further illustrates the invention but, of course, should not be construed as in any way limiting its scope.

### EXAMPLE 1

This example demonstrates that ZFP36L2 regulates hematopoiesis.

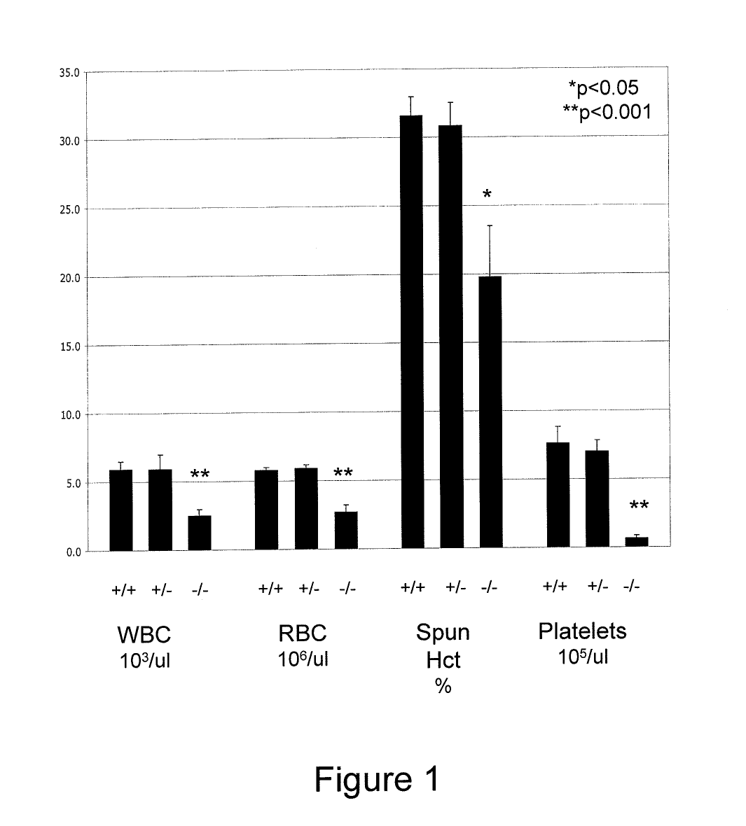

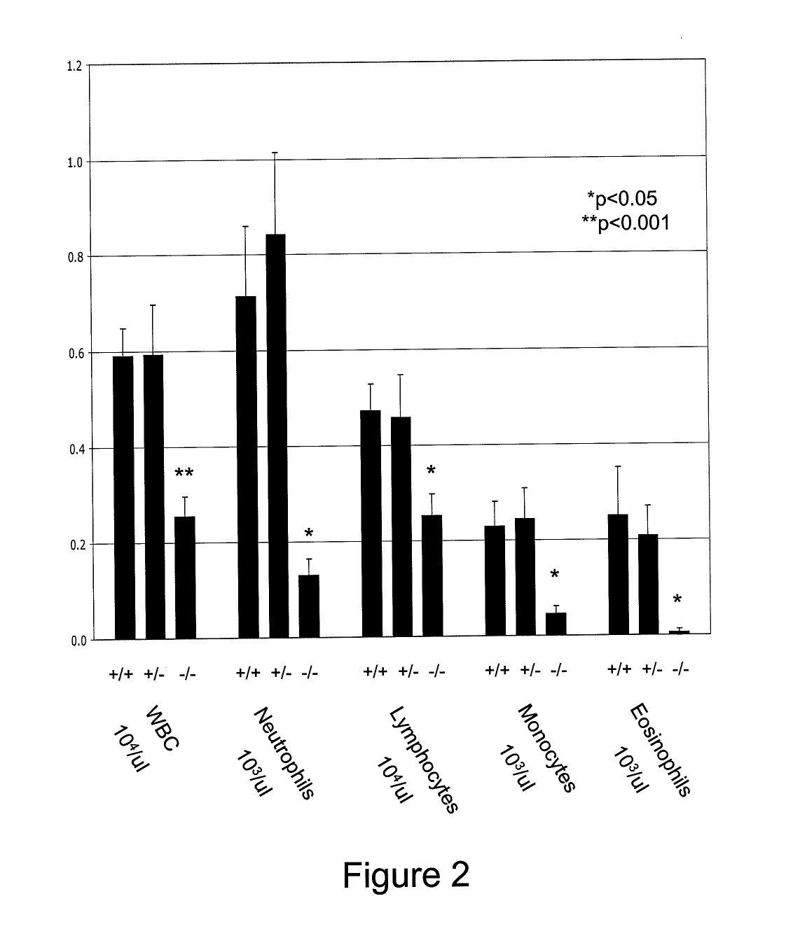

The effect of ZFP36L2 protein on blood counts was analyzed by comparing the complete blood count (CBC) and white blood cell count (WBC) of ZFP36L2 knock-out mice to that of wild-type mice. Peripheral blood was obtained from 2 week old ZFP36L2 wild type mice (6 total) and ZFP36L2 knock-out mice (7 heterozygous and 6 complete knock-out), and the blood was analyzed using a Hemavet 950 hematology analyzer (Drew Scientific, Dallas, Tex.). The results are presented in FIGS. 1 and 2, wherein the results are presented as means±S.E.M. Means were compared by Student's t test.

The results indicate that the ZFP36L2 knock-out mice had significantly lower blood counts than either wild-type or heterozygous mice, indicating that ZFP36L2 expression regulates hematopoiesis.

### EXAMPLE 2

This example further demonstrates that ZFP36L2 regulates hematopoiesis.

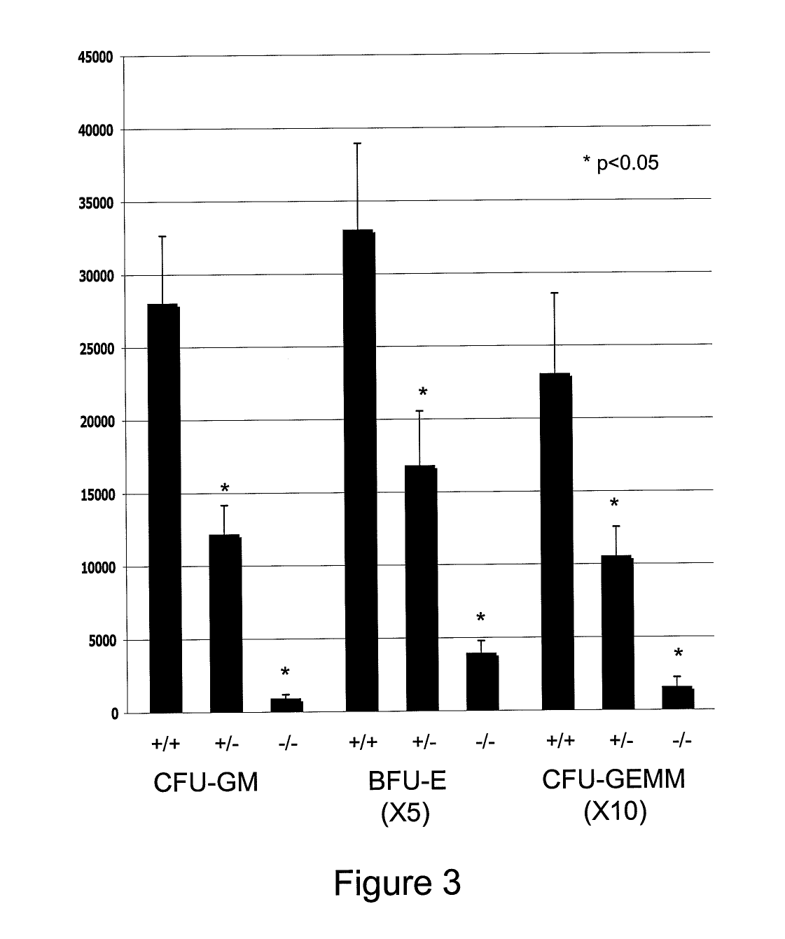

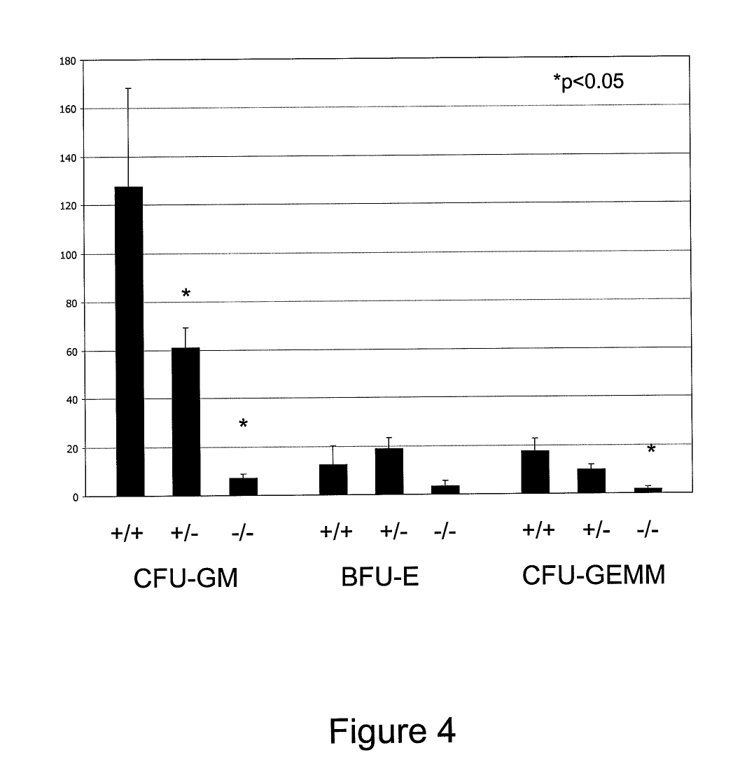

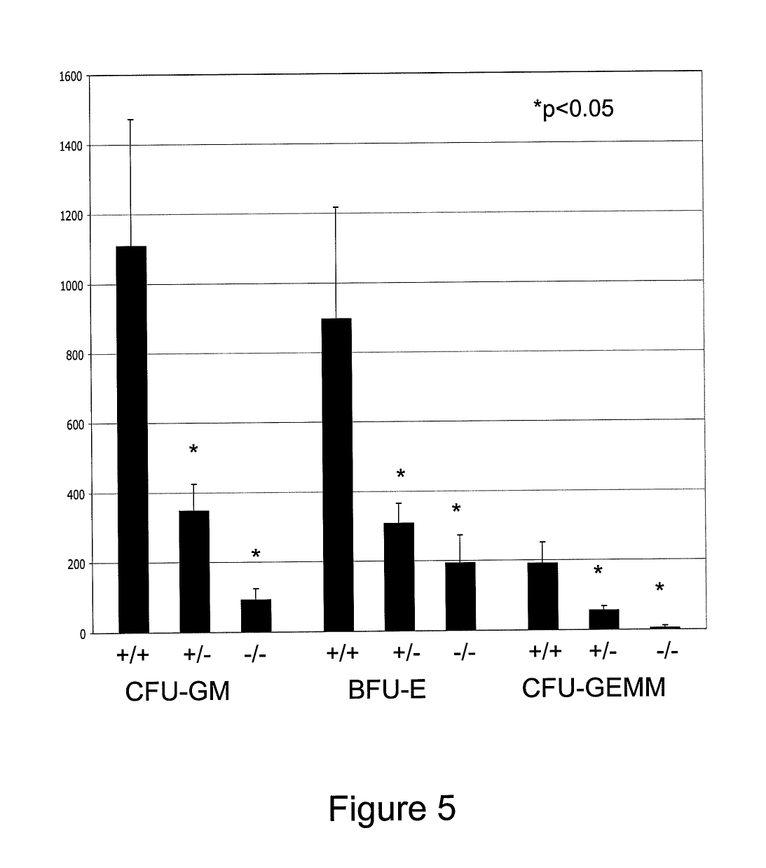

The effect of ZFP36L2 protein on hematopoietic progenitor cells was analyzed by comparing the number of hematopoietic progenitor cells in the fetal livers of wild-type, partial ZFP36L2 knock-out, and complete ZFP36L2 knock-out mice. Assays were performed on fetal livers at embryonic day 14.5 (E14.5; FIG. 3), yolk sacs at embryonic day 11.5 (E11.5; FIG. 4) and the AGM (aorta-gonad-mesonephros) region at embryonic day 11.5 (FIG. 5). Cells from the indicated tissues were plated in 1% methylcellulose culture medium with 30% fetal bovine serum, 0.1 mM hemin, 1 U/ml recombinant human erythropoietin, 5% vol/vol pokeweed mitogen mouse spleen cell-conditioned medium, and 50 ng/ml stem cell factor. Colonies were scored after 7 days incubation at 5% CO₂ at lowered (5%) O₂. Calculations of the absolute numbers of progenitors per organ were based on the nucleated cellularity and colony counts for colony-forming unit—granulocyte, erythrocyte, monocyte, megakaryocyte (CFU-GEMM), burst-forming unit—erythroid (BFU-E), and colony-forming units-granulocyte-macrophage (CFU-GM) for each individually assessed embryo. The results, which are presented in FIGS. 3-5, are expressed as the means±S.E.M. from 8 wild type, 9 heterozygous and 7 knockout embryos (fetal liver), 5 wild type, 10 heterozygous and 5 knockout embryos (yolk sac), and 5 wild type, 13 heterozygous and 6 knockout embryos (AGM regions). Means were compared by Student's t test.

The results show that the ZFP36L2 complete knock-out mice had significantly fewer hematopoietic progenitor cells that either wild-type or heterozygous mice, and the heterozygous mice had, in most categories, significantly fewer hematopoietic progenitor cells than the wild-type mice. These results indicate that ZFP36L2 regulates hematopoiesis.

### EXAMPLE 3

This example further demonstrates that ZFP36L2 expression regulates hematopoiesis.

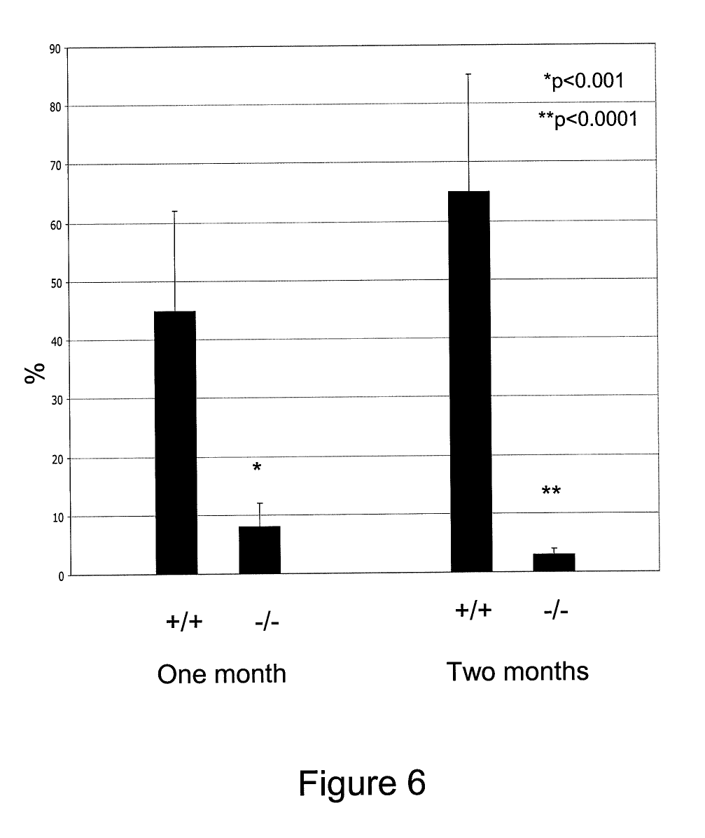

The effect of ZFP36L2 protein on the competitive repopulation of fetal liver cells was analyzed by comparing such repopulation of fetal liver cells from wild type mice with the repopulation of fetal liver cells from ZFP36L2 knock-out mice. Lethally irradiated mice were reconstituted with E14.5 fetal liver cells isolated from either wild type or knockout embryos. Five thousand mutant cells were mixed with 500,000 competitor cells (from a genetically different mouse strain) and injected into the irradiated recipient mouse. Reconstitution of donor-derived cells (percent engraftment) was monitored by staining of blood cells with strain-specific markers at one and two month's post-injection. The results are presented in FIG. 6.

The results show that the cells isolated from the fetal livers of ZFP36L2 knock-out mice repopulated at a significantly lower level than the cells isolated from the fetal livers of wild-type mice. These results indicate that ZFP36L2 regulates hematopoiesis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of detecting abnormal hematopoiesis, or a condition associated therewith, in a subject comprising detecting (a) abnormal ZFP36L2 expression in the subject or (b) a mutation in a ZFP36L2 gene in the subject, wherein (a) or (b) indicates abnormal hematopoiesis or a condition associated therewith.

2. The method of claim 1, wherein the method comprises detecting (a) and wherein abnormally high ZFP36L2 expression indicates abnormally high hematopoiesis or a condition associated therewith.

3. The method of claim 2, wherein the condition associated with abnormally high hematopoiesis is primary or secondary polycythemia, a cancer or tumor, including leukemia, or combination thereof.

4. The method of claim 2, wherein the method further includes treating the subject for a condition associated with abnormally high hematopoiesis.

5. The method of claim 1, wherein the method comprises detecting (a) and wherein abnormally low ZFP36L2 expression indicates abnormally low hematopoiesis.

6. The method of claim 5, wherein the condition associated with abnormally low hematopoiesis is primary or secondary anemia, thrombocytopenia, myelodysplastic syndrome, or a combination thereof.

7. The method of claim 5, wherein the method further includes treating the subject for a condition associated with abnormally low hematopoiesis.

8. The method of claim 1, wherein abnormal ZFP36L2 expression is detected by comparing the level of ZFP36L2 mRNA or ZFP36L2 protein in the subject, or a sample obtained from the subject, to a control.

9. The method of claim 1, wherein the hematopoiesis is of the myeloid lineage.

10. The method of claim 1, wherein abnormal ZFP36L2 expression is detected in a hematopoietic or stromal cell.

11. (canceled)

12. A method of controlling hematopoiesis in a subject that has abnormal hematopoiesis or a condition associated therewith, the method comprising adjusting the level or activity of ZFP36L2 protein in the subject.

13. The method of claim 12, wherein the level or activity of ZFP36L2 protein in the subject is increased.

14. (canceled)

15. (canceled)

16. The method of claim 13, wherein the subject has a condition associated with abnormally low hematopoiesis and wherein hematopoiesis in the subject is stimulated.

17. The method of claim 16, wherein the condition is primary or secondary anemia, thrombocytopenia, myelodysplastic syndrome, or a combination thereof.

18. The method of claim 12, wherein the level or activity of ZFP36L2 protein in the subject is decreased.

19. (canceled)

20. (canceled)

21. The method of claim 18, wherein the subject has a condition associated with abnormally high hematopoiesis and wherein hematopoiesis in the subject is inhibited.

22. The method of claim 21, wherein the condition is primary or secondary polycythemia, a cancer or tumor, including leukemia, or combination thereof.

23. (canceled)

24. (canceled)

25. A method of screening for a compound that modulates hematopoiesis comprising:
(a) administering a test compound to a cell that expresses ZFP36L2, and
(b) detecting a change in ZFP36L2 expression in the cell in the presence of the test compound as compared to a control,
wherein a change in ZFP36L2 expression in the cell indicates that the test compound is likely to modulate hematopoiesis.

26. (canceled)

27. The method of claim 25, wherein a change in the expression of ZFP36L2 is detected by detecting a change in (a′) the level of transcription of the ZFP36L2 gene, (b′) the amount of ZFP36L2 mRNA, (c′) the amount or activity of ZFP36L2 protein.

28. The method claim 25, wherein the cell comprises a nucleic acid encoding ZFP36L2 fused to a nucleic acid encoding a marker protein, and a change in the expression of ZFP36L2 is detected by detecting a change in the level or activity of the marker protein.

29. The method of claim 28, wherein the nucleic acid encoding the marker protein (i) is fused to the 3′ end of ZFP36L2 mRNA, (ii) is fused to a ZFP36L2 promoter, (iii) encodes GFP, (iv) encodes luciferase, or (v) encodes beta-galactosidase.

30. (canceled)

31. (canceled)

32. The method of claim 25, wherein the control is (i) ZFP36L2 expression in the cell, or a cell of the same type, in the absence of the test compound, or (ii) a pre-established baseline ZFP36L2 expression level.

33. The method of claim 25, wherein the method further comprises, prior to administering a test compound to a cell that expresses ZFP36L2, selecting a test compound that binds to (i) ZFP36L2 mRNA, (b) (ii) ZFP36L2 protein, or (iii) ZFP36L2 promoter.

34. (canceled)
